(19) United States
(12) Patent Application Publication
(10) Pub. No.: US 2003/0228299 A1
(43) Pub. Date: Dec. 11, 2003

(54) Use of antioxidant for treating and/or preventing surface ocular disorders

(76) Inventors: Marie-Therese Droy-Lefaix, Hermes (FR); Christophe Baudouin, Paris (FR)

Correspondence Address: DENNISON, SCHULTZ & DOUGHERTY, 1745 JEFFERSON DAVIS HIGHWAY, ARLINGTON, VA, 22202, US

(21) Appl. No.: 10/343,840

(22) PCT Filed: Jun. 7, 2002
(86) PCT No.: PCT/FR02/01955

(51) Int. Cl. A61K 38/44; A61K 31/385

(52) U.S. Cl. 424/94.4; 514/440

(57) ABSTRACT

The invention relates to a pharmaceutical composition for the treatment of superficial eye disorders.

According to the invention, an antioxidant, selected from the group comprising SODs, SOD mimetics and derivatives, racemic alpha-lipoic acid and its R⁺ or R⁻ enantiomer, and mixtures of these compounds, is used for the manufacture of a drug for the treatment and/or prevention of superficial eye disorders.

The invention is applied in human and veterinary medicine.

The invention relates to the use of an antioxidant, selected from the group comprising superoxide dismutases (SODs), superoxide dismutase mimetics, superoxide dismutase derivatives, racemic alpha-lipoic acid, the enantiomers of alpha-lipoic acid, and mixtures of these antioxidants, for the manufacture of a drug for the treatment and/or prevention of superficial eye disorders in humans or animals.

The ocular surface is the transitional mucosa between the deep ocular medium and the external environment. The integrity of the cornea—a transparent ocular window open on the outside for transmitting images to the retina—must be maintained since the loss of its transparency is tantamount to blindness, which is sometimes permanent.

The ocular surface has to be seen as an anatomical and functional barrier protecting the eye from external aggressors. It is formed of a three-part system, namely the cornea, the lacrimal film and the conjunctiva (Hoang-Xuan T. 1998, Pouliquen Y. 1991, Rigal D. 1993, Baudouin Ch. 1998).

Through its anatomical structure and the quality of the interface with the lacrimal film, the corneal epithelium performs a barrier function necessary for protecting the underlying corneal constituents and the intraocular medium.

The lacrimal film consists of three layers: a 0.1 μm thick lipid layer, a 7 μm aqueous intermediate layer and a mucus-rich deep layer. This mucus, secreted by the caliciform cells present inside the conjunctival epithelium, spreads over the ocular surface. It is in contact with the apical cytoplasmic membrane of the superficial cells via the glycocalyx.

The lacrimal film acts by way of physicochemical and immunological properties. Through blinking, it continually drains and eliminates the micro-organisms, foreign bodies and desquamated epithelial cells present on the corneal surface by coating them in the mucosal film of the inferior conjunctival cul-de-sac. This beneficial lacrimation contributes to sweeping and thoroughly cleaning the corneo-conjunctival surface by preventing the adhesion of any germs present.

The mechanical role of the lacrimal flow is coupled with the intervention of the pH and its variations and the intervention of the temperature and the osmolarity of the tears. The tears actively participate in the non-specific defense means of the cornea by virtue of the presence of lactoferrin, lysozyme and immunoglobulins. These non-specific means provided by the tears are coupled with the specific components of the conjunctival tissue.

The complex made up of lacrimal mucus, glycolipids and glycoproteins of the epithelial plasma membranes governs the quality of the corneal surface. In the absence of the lacrimal film, the epithelial surface is hydrophobic and the aqueous phase of the tears deposits as drops rather than spreading. By lowering the surface tension, mucin enables the aqueous component of the tears to spread and to be adsorbed by the epithelial surface, thereby maintaining a stable lacrimal film between successive blinks.

Any anomaly of the mucosal layer will compromise the stability of the lacrimal film and cause it to rupture, forming dry spots; this happens very quickly after the resurfacing effect of blinking. By increasing the adhesion of the lacrimal film, the apical expansions, such as the microvilli and the microplicae of the superficial cells, thus contribute to a better stability of the lacrimal film of tears.

Degradation of this comeo-conjunctival surface favors the adhesion of bacteria on the epithelial surfaces.

To protect itself, the cornea also has the conjunctiva. The conjunctiva is a vascularized mucous membrane covering the surface of the eyeball and of the posterior face of the upper and lower eyelids. It is responsible for the secretion of mucus that is essential for the stability of the lacrimal film and the transparency of the cornea.

It plays an important defense role and is richly vascularized. It contains numerous immunocompetent cells capable of initiating an inflammatory reaction, participating in it and synthesizing immunoglobulins. Furthermore, the morpho-logical characteristics such as the microvilli and the biochemical characteristics such as the enzymatic activity of the epithelial cells enable them to envelop and phagocyte foreign particles like viruses (Hoang-Xuan T. et al. 1998).

Consequently, the three-part system of cornea/conjunctiva/lacrimal film, which represents a system of extreme physiological richness, is capable of becoming inflamed following:

aggression towards the ocular surface by environmental agents,

qualitative or quantitative degradation of the lacrimal film (dry eye syndrome),

an allergy, or

a chronic infection of the ocular surface.

This inflammation causes the release of free radicals, which are very reactive chemical species (Lemp M. A. 1999, Savoure N. 1993). These free radicals cause ocular dryness by a direct effect on the lacrimal film and the cornea.

In the pathology of the ocular surface, oxidative stress occupies a major position by virtue of oxygenated free radicals, which are highly toxic species for the mucosae because of their high content of polyunsaturated fatty acids.

Free radicals are chemical species with a single unpaired electron in their outermost shell, which gives them a high degree of instability compared with the presence of two electrons in the outermost shell (Pryor W. A. 1986, Savoure N. 1993).

The primary radicals are superoxide anions (O₂.⁻), which are a starting point of free radical chains from molecular oxygen. The reduction of molecular oxygen (O₂) by the gain of an electron culminates in the release of a superoxide anion (O₂.⁻), an aggressive form of oxygen. Via a dismutation reaction, the superoxide anion (O₂.⁻) releases hydrogen peroxide (H₂O₂) into the medium. H₂O₂ is then converted to the hydroxyl radical (OH.), a very reactive species. This reaction requires the presence of transition metals such as iron in the ferrous state, or copper. Hemoglobin would be a large provider of ferric iron in free radical oxidation.

Nitrogen monoxide (NO.) is a simple molecule and a free radical centered on the nitrogen atom. It is formed in vivo by the constitutive NO synthases (NOSs), but under certain pathological conditions another NOS, inducible NOS, can be expressed.

In the presence of molecular oxygen (O₂), nitrogen monoxide (NO.) forms nitrogen dioxide (NO₂), which reacts with one molecule of NO to produce dinitrogen trioxide (N₂O₃). NO₂ and N₂O₃ are described as reactive nitrogen species responsible for nitrosative stress.

Furthermore, the superoxide anion (O₂.⁻) can react with nitrogen monoxide (NO.) to form the peroxynitrite anion (ONOO⁻), which is a powerful oxidizing agent capable of inducing oxidative stress leading to the oxidation of numerous cellular targets (Beckman J. S. and Koppenol W. H. 1996, Squadrito G. L. and Pryor W. A. 1998).

The secondary radicals are derived from the membranous lipid peroxidation of polyunsaturated fatty acids (for example arachidonic acid), which are the most sensitive targets. This lipid peroxidation is a chain reaction initiated by the hydroxyl radical (OH.) with the hydrocarbon chain of a polyunsaturated fatty acid (L) to form an alkyl radical (L.). In the presence of molecular oxygen (O₂), the alkyl radicals (L.) cause the release of peroxy radicals (ROO.) and alkoxy radicals (RO.) in the course of the propagation reactions. The presence of transition metals such as iron and copper enables these propagation reactions to be sustained. These peroxides are the cause of a deep disorganization of the membranous architecture, with serious consequences for the molecular interactions (Fridovich I. 1997).

The oxidation of proteins is principally initiated by the hydroxyl radical (OH.), but also depends on the presence of the superoxide anion (O₂.⁻). The oxidative attacks are directed at the side groups or at the asymmetric carbon of amino acids.

The formation of carbonyl groups by the direct oxidation of amino acids is considered to be one of the major oxidative modifications of proteins. These carbonyl groups are used as protein oxidation markers (Dean R. T. et al. 1997).

DNA bases are very sensitive to the action of biological oxidizing agents. The superoxide anion (O₂.⁻) can oxidize DNA by way of the formation of the hydroxyl radical (OH.) in the presence of iron (Henle E. S. and Linn S. 1997).

Consequently, an increased release of free radicals adversely affects the quality of the tears by degrading the serum proteins such as albumin, lysozyme, lactoferrin, immunoglobulins and glycoproteins of the mucus. The free radicals thus have deleterious effects on the cells of the cornea and conjunctiva, ranging from the presence of erosions to severe and very disabling ulcerations.

Environmental factors are permanent sources of attack on the ocular surface.

In this domain, bacteria, viruses, foreign bodies, the substantial evaporation of tears caused by extensive computer work, and atmospheric pollutants from photochemical smogs are large providers of free radicals.

Every year tens of thousands of patients complain of the deleterious effects of these factors on the ocular surface. Thus more than 10 million people in the USA, 15% of the population aged 65, suffer from aggression towards the ocular surface.

From the clinical point of view, ocular dryness is responsible for often neglected sensations of eye fatigue, a simple need to blink, smarting and foreign bodies, to more severe sensations of burning, pain and, should painful superficial keratitis appear, visual defects due to the induced epitheliopathy, and opacification of the cornea.

The severity of the dryness allows the following distinction in clinical practice:

simple dry syndromes caused by environmental factors, promoted by an underlying condition such as the menopause, and worsened by an aggressive environment (Azzurolo A. et al. 1995),

chronic disease of the ocular surface, namely dry keratoconjunctivitis, where chronic cellular distress culminates in true and often inextricable pathogenic vicious circles.

In superficial eye disorders, the two major physiopathological developments are the immuno-inflammatory component of the attack on the eye, and the apoptotic phenomena induced both by inflammation and by certain eyewashes (Sullivan D. A. et al. 1999).

Despite its often intense symptomatology, its unstable lacrimal film and sometimes its keratitis, dry eye is still a white eye that is very different from the red eye of chronic conjunctivitis. However, chronic inflammation is always omnipresent in dry eye, be it a primary inflammation, as in Gougerot-Sjögren disease, an inflammation secondary to dry keratitis, an inflammation associated with an eye allergy, a viral infection, blepharitis or rosacea, or an iatrogenic inflammation.

Gougerot-Sjögren disease is one of the most characteristic forms of inflammatory dryness. It affects not only the principal lacrimal gland, culminating in its fibrosis, but also the whole of the conjunctival surface, which bathes in a medium rich in inflammatory mediators. Local activation of the immune system causes the conjunctiva to be infiltrated by inflammatory cells and modifies the metabolism of the epithelial cells. These then express immune markers and secrete cytokines and free radicals. It is this generalized inflammation that makes Gougerot-Sjögren disease serious (Jones D. T. et al. 1998).

The induction of a dry syndrome by a viral keratoconjunctivitis or as the consequence of an acute or subacute conjunctivitis is a frequent occurrence. These disorders result in a sometimes permanent loss of mucosal cells on the conjunctival surface. Even after treatment with eyewashes, patients complain of lingering symptoms similar to the sensations experienced during the initial disease. They believe that the disease has not been treated, whereas in fact they are suffering from a secondary dryness that is often aggravated by continuous aggressive treatments, trapping the patients in a vicious circle.

Likewise, chronic infections are frequently responsible for severe ocular dryness, which is all the more difficult to treat because the signs of infection are rather unspecific, hidden or unrecognized (Hoang-Xuant T. et al. 1998).

Blepharitis and ocular rosacea are also frequent causes of qualitative ocular dryness, with infectious and inflammatory components. These disorders cause a dysfunction of the meibomian glands, which disturbs the composition of the lipid phase of the lacrimal film and increases the rate of evaporation of the tears. These phenomena manifest themselves clinically as a shortening of the BUT (tear stretching resistance) and cause an epithelial hyperosmolarity. These meibomian dysfunctions result in cellular distress, a rupturing of the intercellular junctions, a loss of caliciform cells and probably a deficient secretion of mucus by the conjunctival cells (Toda I. et al. 1995).

A banal dryness is observed by a degenerative atrophy of the lacrimal glands, which is treated with eyewashes that are incapable of relieving the patients. A toxic effect can be observed in the places where irritant eyewashes accumulate, particularly in the area of the inferior palpebral or nasal fissure in patients presenting with open-angle glaucoma or secondary glaucoma the use of preservatives such as benzalkonium chloride, i.e. quaternary ammonium compounds, in eyewashes. These preservatives also have a significant degree of toxicity by way of the production of free radicals. This results in a reduction of the tear stretching resistance, which is a direct toxicity for the epithelial cells, with epithelial erosions and an inflammatory reaction. It must be pointed out that the half-life of a benzalkonium compound is 20 hours and that significant levels of preservatives still remain 168 hours after the instillation of a single drop (De Saint Jean et al. 1999).

Consequently, whether it be primary or secondary, the inflammatory reaction is an essential component of dry eye syndromes. It is therefore necessary to develop treatments suitable for dry eye syndrome and much more generally for superficial eye disorders.

Now, at the present time, the aim of all the proposed treatments is to alleviate the symptoms of superficial eye disorders, i.e. ocular dryness, and not really to cure the cause of the disorder that is giving rise to this ocular dryness.

Thus, as soon as the treatment stops, the symptoms reappear.

Furthermore, no preventive treatment has been proposed for avoiding the appearance of an inflammation of the ocular surface when it is known that it is going to be exposed to inflammatory agents, for example the preservatives present in compositions to be instilled into the eye for the treatment of various pathological conditions.

In fact, the administration of topical products containing preservatives (antiseptic substances) to the ocular surface can modify its equilibrium and cause serious anomalies of the conjunctiva and cornea that are capable of developing inconspicuously and only manifesting themselves very much later, sometimes totally unexpectedly.

The toxic effects of quaternary ammonium compounds have been studied the most.

Benzalkonium chloride (BAC) is present in virtually all multiple-dose eyewashes, including those of antiglaucomatous eyewashes. Even in very low doses, it induces cellular apoptosis with the release of oxygenated free radicals. It substantially degrades the corneal epithelium and stimulates infiltration of the conjunctiva by inflammatory cells.

Furthermore, racemic alpha-lipoic acid or its R⁺ or R⁻ enantiomer is an antioxidant which has been used especially for protection against UV, peripheral neuropathies and certain lipodystrophies and for slowing down the replication of HIV.

It has also been used in combination with numerous other active ingredients in oral compositions for its protective effects on the retina, in age-related degeneration, glaucoma and increased ocular pressure, i.e. for disorders not of the ocular surface but of the posterior segment of the eye.

As regards superoxide dismutases, hereafter called SODs, these are enzymes present in organisms at the extracellular and intracellular level.

Cu/Zn SODs are used in cosmetics and for the treatment of cancers, in which case they are administered intravenously.

The object of the invention is to afford not only a basic treatment of superficial eye disorders capable of causing ocular dryness, but also the prevention of these disorders.

“Basic treatment” is to be understood as meaning a true cure and not simply a treatment of the symptoms due to the eye disorder, such as ocular dryness.

To this end the invention proposes the use of an antioxidant, selected from the group comprising superoxide dismutases (SODs), superoxide dismutase mimetics, superoxide dismutase derivatives, racemic alpha-lipoic acid, the R⁺ enantiomer of alpha-lipoic acid, the R⁻ enantiomer of alpha-lipoic acid, and mixtures of these compounds, for the manufacture of a drug for the treatment and/or prevention of superficial eye disorders in humans or animals.

In a first embodiment, the antioxidant is an SOD and/or an SOD derivative and/or mimetic.

In this first embodiment, the SOD is preferably a wheat SOD.

However, whatever the SOD or SOD mimetic or derivative used, the SOD is preferably present in the drug at a concentration of between 1 μg/ml and 500 μg/ml, preferably of between 25 μg/ml and 50 μg/ml.

In a second, particularly preferred embodiment, the antioxidant is racemic alpha-lipoic acid and/or its R⁺ or R⁻ enantiomer.

However, it is preferable to use racemic alpha-lipoic acid in this embodiment.

In all cases the racemic alpha-lipoic acid or its R⁺ or R⁻ enantiomer is preferably present in the drug at a concentration of between 0.05 μg/ml and by 200 g/ml, preferably of between 0.5 μg/ml and 5 μg/ml.

In a third embodiment, also particularly preferred, the antioxidant is a mixture of racemic alpha-lipoic acid and/or its R⁺ or R⁻ enantiomer, and one or more SODs and/or an SOD derivative and/or mimetic.

In this embodiment the alpha-lipoic acid and/or its R⁺ or R⁻ enantiomer is preferably present in the drug at a concentration of 0.05 μg/ml to 200 μg/ml, preferably of 0.5 μg/ml to 5 μg/ml, and the SOD and/or SOD derivative and/or mimetic is preferably present in the drug at a concentration of between 1 μg/ml and 500 μg/ml, preferably of between 25 μg/ml and 50 μg/ml.

In all the embodiments of the invention, the superficial eye disorders treated are those which cause ocular dryness.

More particularly, the superficial eye disorders treated can be a degradation of corneal cells.

The eye disorders treated can also be due to exposure to environmental agents such as ozone, nitrogen oxide, sulfur dioxide, volatile organic compounds and particles released by diesel engines.

They may also have been caused by prolonged exposure to a computer screen, a television screen or a video monitor.

In addition, the superficial eye disorders treated are those due to the wearing of lenses.

As a further possibility, the eye disorders treated are those due to exposure to salt water and/or chlorinated water.

These eye disorders can also be due to exposure to ultraviolet A and/or B and/or C or to X-rays.

The eye disorders treated can also be due to exposure to bacteria, viruses and/or fungi.

Gougerot-Sjögren disease, blepharitis and ocular rosacea can also be treated.

Superficial eye disorders due to exposure to allergens, associated with an inflammatory reaction and a degradation of the ocular surface, can also be treated.

In particular, eye disorders due to exposure to one or more preservatives present in compositions administered topically to the eye are not only treated, but can also be prevented by virtue of the invention, more particularly when the preservative is benzalkonium chloride.

The drug manufactured by virtue of the invention can be in the form of an eyewash, an ointment or a gel and can also contain pharmaceutically acceptable excipients.

The invention will be understood more clearly and other objects, advantages and characteristics thereof will become more clearly apparent from the following explanatory description referring to the Figures, in which:

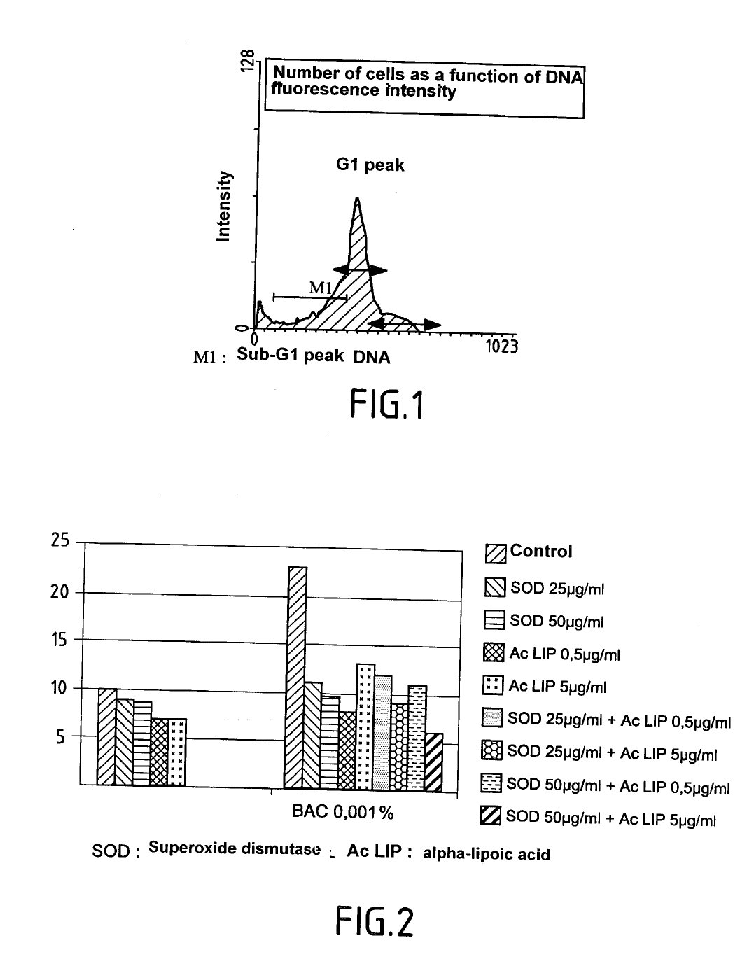

FIG. 1 shows a fluorescence peak which is typical and representative of the information of conjunctival cells and is emitted by the DNA of conjunctival cells, this peak being obtained by flux cytofluorimetric analysis, and

FIG. 2 shows, in the form of histograms, the results of the analysis of tests performed with the compounds of the invention.

The superficial eye disorders to which the invention relates can be due . . . :

exposure to environmental agents such as photochemical smogs in towns (ozone, nitrogen oxides, sulfur dioxide, volatile organic compounds, diesel particles),

prolonged exposure to a computer, television or video monitor screen,

the wearing of contact lenses,

exposure to salt water or chlorinated water,

exposure to ultraviolet (UV A, B, C), X-rays, γ-rays or radioactive radiation,

exposure to environmental agents such as bacteria, viruses or fungi,

a degradation of the ocular surface chosen from Gougerot-Sjögren disease, blepharitis and ocular rosacea,

exposure to allergens, associated with an inflammatory reaction and a degradation of the ocular surface, or

exposure to the preservatives contained in compositions to be instilled into the eye.

The treatment of these various disorders has hitherto been limited to a symptomatic treatment consisting in relieving the human or animal patient by proposing the local application of various eyewashes, gels or ointments in order to alleviate ocular dryness and other symptoms of discomfort resulting from these disorders.

However, no basic treatment, i.e. no treatment capable of really curing and healing the ocular surface, has been proposed hitherto. No preventive treatment has ever been proposed either.

Now, it has been discovered that antioxidants are active compounds for a basic treatment of superficial eye disorders.

The pharmaceutical composition obtained by using these antioxidants will therefore contain one or more antioxidants as active ingredient, together with pharmaceutical excipients acceptable for local application to the eye.

Particularly appropriate antioxidants are superoxide dismutases, superoxide dismutase mimetics, superoxide dismutase derivatives, racemic alpha-lipoic acid or its R⁺ or R⁻ enantiomer, and mixtures of these compounds.

Superoxide dismutases, also called SODs, represent one of the three main classes of antioxidizing enzymes in the organism, along with catalase and glutathion peroxidase (Fridovich 1986).

Superoxide dismutases, or SODs, are antioxidizing metalloenzymes discovered in 1969 by McCord and Fridovich; they are capable of catalyzing the dismutation reaction of the superoxide anion (O₂.⁻) to hydrogen peroxide (H₂O₂) and oxygen in an aqueous medium according to the following reaction, which requires the presence of iron:

2O₂.⁺+2H⁺H₂O₂+O₂ (1)

Fe²⁺+H₂O₂→Fe³⁺+OH. (Fenton's reaction) (2)

Superoxide dismutases are present in organisms at the extracellular and intracellular level.

They are divided into 3 groups:

Cu/Zn SODs,

Mn SODs,

Fe SODs.

They can be of human origin, in which case one refers to homologous SODs, or they can be of animal, vegetable or bacterial origin, in which case one refers to heterologous SODs.

They can be either extracted or recombinant or synthetic.

Their effects can be mimicked by various complexes called superoxide dismutase mimetics.

The role of endogenous SODs is to assure the protection of cells and extracellular spaces against aggression by superoxide anions (O₂.⁻). Under normal conditions, the levels of SODs, and the specific activity of the enzyme, are relatively constant for one and the same tissue in one and the same healthy individual (Michelson A. M. 1987).

In cases of oxidative stress due to the high production of free radicals and the resulting lipid peroxidation, the levels of SODs are modified. Pathological conditions, such as acute or chronic inflammations and autoimmune diseases, indicate the disappearance of the adaptive qualities of SODs. Very low levels of SODs have been reported in pathological conditions of this type.

Three types of endogenous SODs have so far been described, namely Cu/Zn SODs normally located in the cytosol of eukaryotic cells, in the extracellular fluid of mammals and in certain bacteria, Mn SODs in prokaryotes or in mitochondria, and Fe SODs, containing iron, located in anaerobic bacteria and in prokaryotes.

Cu/Zn SODs are subdivided into Cu/Zn SODs I and II.

There currently exist human Cu/Zn SOD in recombinant form and vegetable Cu/Zn SODs that are either extracted from wheat, melon, tomato, pollen, spinach or rice, or recombinant.

There also exist extracted bacterial Cu/Zn SODs, including that of Saccharomyces cerevisiae, animal Cu/Zn SODs of bovine extraction and recombinant Cu/Zn SODs.

Mn SODs are subdivided into Mn SODs I and II, which exist in extracted or recombinant form.

Likewise, Fe SODs exist in extracted or recombinant form.

Among these SODs, Cu/Zn SODs are more particularly preferred for use in the invention.

SODs are enzymes with a molecular weight of 32 kd. They are composed of two subunits, each containing a copper atom and a zinc atom bonded non-covalently (Keller G. A. et al. 1991).

Exogenous SODs exist in native form, of human extraction (homologous protein) or animal or vegetable extraction (heterologous proteins), or in recombinant form. To extend their half-lives and improve their distribution at the cellular level, it is sometimes of interest to vectorize them without, however, affecting their pharmacological properties.

Complexes which mimic the effect of the various SODs at the extracellular or intracellular level, i.e. SOD mimetics, are also particularly preferred compounds in the invention because they have a better permeability and stability.

Furthermore, like SODs of natural origin, they catalyze the superoxide anion, but they also inhibit xanthine oxidases.

Particularly preferred among these mimetics are monomeric trans-bis(naproxenato)bis(3-pyridylmethanol)copper(II), complexes derived from bioflavonoids, like rutin, that contain iron and copper, 1,4,7,10,13-pentoazacyclo-pentadecane and SODase.

In various tests performed in vitro and in vivo, heterologous SODs are found to have a significantly higher antiinflammatory activity than homologous SODs.

These heterologous SODs, which have a low incidence of an immunological nature, do not induce a proinflammatory reaction and protect the cells from degeneration by inhibiting the formation of oxygenated free radicals.

Consequently, because of the beneficial effects of SOD, the present invention proposes to use it by itself or in the presence of a pharmaceutical vehicle in the treatment of superficial eye disorders.

However, SODs are molecules which are rapidly eliminated from the cornea by the tears, so they cannot exert their action fully when instilled into the eye.

Also, these are giant molecules which diffuse poorly into the cellular tissues.

Consequently, in a second embodiment, the invention proposes the use of alpha-lipoic acid and/or its R⁺ or R⁻ enantiomer as an antioxidant.

Alpha-lipoic acid (LA), discovered in 1937 and chemically characterized by Lester Reed in 1951, is known by a variety of names, including 2-dithiolane-3-pentanoic acid, 1,2-dithiolane-3-valeric acid and thioctic acid. It is an antioxidant which is synthesized by the human body but is also present in small amounts in potatoes, spinach and red meat.

The production of LA, like that of other antioxidants, declines with age in humans.

It has a chain of eight carbons and a heterocycle with two adjacent sulfur atoms which is capable of donating one or two hydrogen atoms very easily.

This alpha-lipoic acid is a racemic form (RAC-alpha-lipoic acid, DL-alpha-lipoic acid), but it is also active as its R⁺ or R⁻ enantiomer.

The most active form is R⁺-alpha-lipoic acid, or R-thioctic acid, or R-alpha-lipoic acid, or dexlipotam 1200 22 2.

Alpha-lipoic acid and its derivatives can thus take the form of a multiplicity of chemical complexes.

It is rapidly absorbed by the cells and transformed to dihydrolipoate (DHLA), an extremely powerful reducing agent. The latter possesses two sulfur-containing groups that enable it to donate one or two hydrogen atoms very easily.

LA/DHLA is capable of:

trapping free radicals (hydroxyl radical OH., hypochlorous acid HClO, singlet oxygen O₂);

chelating metals (stable complexes with Cu²⁺, Mn²⁺ and Zn²⁺);

regenerating the oxidized forms of vitamins E and C, glutathion and thioredoxin; in its presence, an increase in glutathion is observed in the cells;

inhibiting the expression of genes (NF-KB factor during an inflammatory response).

DHLA also protects against the deleterious effects of ischemia-reperfusion by inhibiting the activity of xanthine oxidase, an enzyme which releases the superoxide anion (Prenn J. H. et al. 1990, 1992, Scheer B. and Zimmer G. 1993, Serbinova E. et al. 1992).

For these reasons, racemics alpha-lipoic acid and its enantiomers are particularly advantageous active ingredients for the treatment and/or prevention of superficial eye disorders in humans or animals.

As the active ingredient it will form part of the composition of a gel, eyewash or ointment, optionally containing pharmaceutically acceptable excipients, for the treatment of these superficial disorders.

Although alpha-lipoic acid and its enantiomers, by themselves or in combination with one another, on the one hand, and superoxide dismutases, by themselves or in combination with one another, on the other hand, have proved to be excellent active principles for the treatment of superficial eye disorders in humans and animals, it is advantageous to use alpha-lipoic acid in combination with one or more of the superoxide dismutases mentioned above.

In fact, in vitro studies showed that SODs and alpha-lipoic acid, at different concentrations, when introduced into a pharmaceutical composition, are particularly effective active ingredients for the treatment or prevention of superficial eye disorders.

Other in vitro studies were conducted in which different concentrations of alpha-lipoic acid and different superoxide dismutases were tested using different techniques for exploring the ocular surface, closely complementing clinical research.

The following tests were thus carried out.

In a first stage, different concentrations of the active compounds of the invention (Cu/Zn SOD, alpha-lipoic acid) are tested using different techniques for exploring the ocular surface, closely complementing clinical research.

Evaluation of Oxygenated Free Radicals

The tests are performed by cytofluorimetric microtitration in cold light. By purifying the thermal energy of the illumination system (infrared and Joule effect), this novel technique makes it possible to obtain a very low background noise and hence to have a good signal-to-noise ratio and a good sensitivity. It combines the reproducibility of the microplate methods with the sensitivity of the cytometric methods and has a very broad detection spectrum (280-870 nm). The microplate is scanned by a pencil of light of a given wavelength which illuminates each culture well for less than 0.3 second, thereby limiting any probe extinction phenomenon.

Dichlorofluorescein Diacetate (DCFH-DA) Test

Corneal and/or conjunctival cells of human origin, in culture, are exposed to various environmental agents in order to induce the release of free radicals. The active compounds of the invention (Cu/Zn SOD or alpha-lipoic acid) are tested at different concentrations on this model. The presence of free radicals in the culture medium is evaluated by the DCFH-DA test (Molecular Probes, Eugene, Oreg., USA) in fluorimetry (λexc.=490 nm−rem.=535 nm, I=4 V). The fluorogenic probe becomes fluorescent when it binds to hydrogen peroxide. The DCFH-DA solution used is a 20 μM solution in Dulbecco-modified minimum essential medium (DMEM). The cultures are then placed for 20 minutes in DMEM containing the probe, after which they are extracted in order to measure the production of hydrogen peroxide. The measurement is made directly on the cells in 96-well plates (Debbasch C. et al. 2000).

The results show that, with the active compounds tested (Cu/Zn SOD or alpha-lipoic acid), there is a significant dose-related decrease in the deleterious effects of free radicals on corneal and/or conjunctival cells, protecting them from membranous damage and apoptotic phenomena. Apoptosis is programmed cell death.

Hydroethidine Test

Corneal and conjunctival cells of human origin, in culture, exposed to various aggressors in order to induce the release of free radicals, are treated with the active compounds of the present invention (Cu/Zn SOD or alpha-lipoic acid) at different concentrations, or are left untreated.

Hydroethidine (Molecular Probes) is a fluorogenic probe which is oxidized to the ethidium cation in the presence of the superoxide anion. This probe becomes fluorescent when it binds to the superoxide anion (λexc.=490 nm−λem.=600 nm, I=6 V). The hydroethidine solution used is a 5 μM solution. The cell cultures are placed for 10 minutes in DMEM containing the probe, after which the cells are extracted in order to measure the production of superoxide anion. The measurement is made in 96-well microplates.

The analyses are thus performed on 5000 cells per well, each measurement being repeated six times. The results are expressed in fluorescence units. The mean fluorescence values are calculated in each group and compared by a Mann-Whitney non-parametric U-test.

The results observed confirm those of the DCFH-DA test. In the presence of the active compounds (Cu/Zn SOD or alpha-lipoic acid), a significant dose-related reduction is observed in the production of superoxide anion and in the cellular degeneration associated with lipid peroxidation of the membranes.

Effects on Benzalkonium Chloride

Demonstration, on conjunctival cells of human origin, in culture, of a protective effect of the products of the invention (Cu/Zn SOD or alpha-lipoic acid) on the necrosis induced by preservatives such as quaternary ammonium compounds (benzalkonium chloride) that are responsible for the production of free radicals

Quaternary ammonium compounds, which are recognized for inducing a qualitative ocular dryness, in fact have detergent properties which modify the lipid phase of the lacrimal film and accelerate its evaporation. They degrade the epithelial microvilli and thus oppose the attachment of the mucus, contributing to an additional instability of the lacrimal film. At low concentration (0.004%) they reduce the rupture time and cause direct toxicity on the superficial cells by way of epithelial erosions, which, depending on the concentration, can range from apoptotic degeneration to true necrosis (Baudouin et al. 1991, De Saint Jean M. et al. 1999).

In relation to the dose, the active compounds of the invention significantly reduce the toxicity of benzalkonium chloride on conjunctival cells in culture. By inhibiting the production of free radical species by this detergent, the active compounds of the invention reduce the epithelial erosions.

Now, quaternary ammonium compounds, and particularly benzalkonium chloride, are present as preservatives in a large majority of eyewashes, eye gels and eye ointments.

The compounds of the invention, which make it possible to thwart the adverse side effects of these preservatives, may therefore be used not only as active principles for curing already established superficial eye disorders, but also as additives to other active principles for preventing the side effects of the preservatives present in the pharmaceutical composition administered, said preservatives being both those of the quaternary ammonium type and any other preservatives capable of causing the formation of oxygenated free radicals and hence of causing apoptotic phenomena.

In particular, the incorporation of these compounds as additives in eyewashes for the treatment of glaucoma is particularly recommendable.

Thus SODs, alpha-lipoic acid and mixtures thereof are useful both for curing and for preventing superficial eye disorders.

The advantageous effects of the compounds of the invention are clearly proven by the results of the following tests.

### EXAMPLE

The apoptosis induced by benzalkonium chloride was chosen as the model of superficial eye disorders treated and/or prevented by the invention.

The effects of:

an HP wheat SOD marketed by Laboratoires SILAB,

racemic alpha-lipoic acid supplied by Laboratoires LALILAB, Inc., and

mixtures of this SOD and this alpha-lipoic acid were analyzed by flux cytofluorimetry on continuous-line conjunctival cells (Wong-Kilbourne derivative of Chang conjunctiva, clone 1-5C-4, ATCC CCL-20.2) by means of the percentage of cellular DNA in the sub-G1 phase, which represents the extent of the apoptosis induced by benzalkonium chloride and hence the extent of cellular aggression.

This study of apoptosis is conducted using flux cytofluorimetry to compare the percentage of DNA in the sub-G1 phase obtained before and after treatment with the compounds of the invention.

In fact, apoptosis is characterized by a fragmentation of the DNA into identical fragments of 200 base pairs. The cells involved in this process therefore have a reduced DNA content.

This fragmentation of the DNA can be measured in situ by flux cytofluorimetry using the DNA stain propidium iodide. The reduced DNA appears with a lower fluorescence intensity than that of normal cells. Cytofluorimetry gives a peak such as that shown in FIG. 1. As can be seen in FIG. 1, this peak is divided into three zones marked M1, G1 peak, and S and G2M phase. The number of normal cells appears in the G1 peak zone. On the other hand, the cells which have a lower fluorescence intensity, i.e. those whose DNA has been fragmented, appear underneath this G1 peak, i.e. in what is called the sub-G1 peak located in the zone marked M1 in FIG. 1.

The operating conditions of these tests are as follows:

Materials and Methods:

High-purity wheat SOD from Laboratoires SILAB, molecular weight=3500 g, taken by default.

Racemic alpha-lipoic acid from Laboratoires LALILAB, Inc., molecular weight=205.3 g, at 0.5 μg/ml and 5 μg/ml.

Apoptosis was induced by adding 0.001% by volume of benzalkonium chloride (BAC), based on the total volume of the sample, to DMEM (Dulbecco-modified Eagle's medium: reference 21885, Gibco BRL Products; enriched with 10% by volume of fetal calf serum, based on the total volume of the sample, and supplemented with 50 mg/ml of streptomycin and 50 IU/ml of penicillin) in which conjunctival cells are cultivated in 6-well plates.

In practice, the conjunctival cells were cultivated in the culture medium described above and, at 80% confluence, SOD, alpha-lipoic acid and mixtures thereof were added and incubation was carried out for 45 minutes at 37° C. Benzalkonium chloride was then added to each well. After 15 minutes of incubation, the supernatants in each well were collected in 15 ml tubes; the cells were detached by the action of 0.25% trypsin for 5 minutes at 37° C. and added to their respective supernatants. After centrifugation of the tubes, the cells were washed twice in phosphate buffer. The cellular residue was resuspended in phosphate buffer at a concentration of 1 million cells per milliliter.

One hundred microliters of this cellular suspension having a concentration of 10⁶/ml were first fixed and permeabilized in one milliliter of absolute ethanol at −20° C. for one hour; then, after washing in phosphate buffer, the DNA was stained with propidium iodide (100 microliters at a concentration of 0.5 mg/ml) for one hour at 4° C. and subsequently analyzed on a cytofluorimeter (Epics XL-MCL, Beckman).

The percentage of sub-G1 peaks, i.e. the percentage of fragmented DNA, and consequently the number of conjunctival cells degraded by apoptosis, was measured. These measurements were made on:

conjunctival cells cultivated in DMEM only, as control;

conjunctival cells cultivated in DMEM and treated with 25 μg/ml of wheat SOD, as control;

conjunctival cells cultivated in DMEM and treated with 50 μg/ml of wheat SOD, as control;

conjunctival cells cultivated in DMEM and treated with 0.5 μg/ml of alpha-lipoic acid, as control;

conjunctival cells cultivated in DMEM and treated with 5 μg/ml of racemic alpha-lipoic acid, as control;

conjunctival cells cultivated in DMEM and treated with benzalkonium chloride (BAC), as reference: this is to measure the apoptosis induced by benzalkonium chloride in the conjunctival cells; the decrease in apoptosis obtained by means of the invention will be measured by reference to this maximum value;

conjunctival cells cultivated in DMEM (in which apoptosis has been induced with benzalkonium chloride) and then treated with 25 μg/ml of wheat SOD;

conjunctival cells cultivated in DMEM (in which apoptosis has been induced by the addition of benzalkonium chloride (BAC)) and treated with wheat SOD at a concentration of 50 μg/ml;

conjunctival cells cultivated in DMEM (in which apoptosis has been induced by the addition of benzalkonium chloride (BAC)) and treated with 5 μg/ml of racemic alpha-lipoic acid;

conjunctival cells cultivated in MEM (in which apoptosis has been induced by the addition of benzalkonium chloride (BAC)) and treated with a mixture of 50 μg/ml of wheat SOD and 0.5 μg/ml of racemic alpha-lipoic acid; and

conjunctival cells cultivated in MEM (in which apoptosis has been induced by the addition of benzalkonium chloride (BAC)) and treated with a mixture of 50 μg/ml of wheat SOD and 5 μg/ml of racemic alpha-lipoic acid.

The numerical results of these tests are collated in Table 1 below:

TABLE 1
PERCENTAGE OF sub-G1 PEAKS

| | |
|---|---|
| DMEM control | 10 |
| SOD 25 μg/ml DMEM | 9 |
| SOD 50 μg/ml DMEM | 8.6 |
| Alpha-lipoic acid 0.5 μg/ml DMEM | 7 |
| Alpha-lipoic acid 5 μg/ml DMEM | 7 |
| DMEM control + BAC 0.001% | 23 |
| SOD 2.5 μg/ml DMEM + BAC 0.001% | 11 |
| SOD 50 μg/ml DMEM + BAC 0.001% | 9.8 |
| Alpha-lipoic acid 0.5 μg/ml DMEM + BAC 0.001% | 8 |
| Alpha-lipoic acid 5 μg/ml DMEM + BAC 0.001% | 13 |
| SOD 25 μg/ml + alpha-lipoic acid 0.5 μg/ml DMEM + | 12 |
| BAC 0.001% | |
| SOD 25 μg/ml + alpha-lipoic acid 5 μg/ml DMEM + BAC 0.001% | 9 |
| SOD 50 μg/ml + alpha-lipoic acid 0.5 μg/ml DMEM + | 11 |
| BAC 0.001% | |
| SOD 50 μg/ml + alpha-lipoic acid 5 μg/ml DMEM + BAC 0.001% | 6 |

The results are also collated in FIG. 2 in the form of a histogram.

In FIG. 2 the left-hand side shows the histograms corresponding to the controls and the right-hand side shows the histograms corresponding to the cells in which apoptosis has been induced by the addition of benzalkonium chloride (BAC).

It can be seen from Table 1 and FIG. 2 that apoptosis already occurs in the absence of any external triggering factor.

It can be seen from the histograms shown on the right-hand side of FIG. 2 that the compounds of the invention have a favorable action on the apoptosis of conjunctival cells.

It is important to note that the curative effect and hence preventive effect of alpha-lipoic acid is greater than that of SOD at concentrations much lower than those of SOD.

It is also important to note that, whereas the favorable effect of SOD increases with concentration, the opposite occurs with alpha-lipoic acid. It can also been seen from Table 1 and FIG. 2 that there is a synergistic effect between SOD and alpha-lipoic acid, especially when using a mixture containing the lowest concentration of SOD tested and the highest concentration of alpha-lipoic acid, which is surprising insofar as it has been seen that alpha-lipoic acid by itself acts most favorably at low concentration.

One explanation could be that SODs are giant molecules and alpha-lipoic acid is a smaller molecule which would fit into the spaces between the different SOD molecules, thereby forming a particularly effective association with SOD.

Thus, to obtain the beneficial effects of the compounds of the invention for the treatment and/or prevention of superficial eye disorders, pharmaceutical compositions containing between 1 μg/ml and 500 μg/ml of SOD, preferably containing from 25 to 50 μg/ml of SOD, for administration by instillation into the eye, are very effective. However, compositions containing racemic alpha-lipoic acid or one of its enantiomers will advantageously be used at concentrations of between 0.05 μg/ml and 200 μg/ml in a pharmaceutical composition for instillation into the eye. The compositions will preferably contain between 0.05 μg/ml and 5 μg/ml of racemic alpha-lipoic acid and/or one of its enantiomers.

Even more favorably, the compositions for the treatment and/or prevention of superficial eye disorders will contain a mixture of SOD or SOD derivatives or mimetics, at the concentrations mentioned above for SOD by itself, and racemic alpha-lipoic acid or alpha-lipoic acid in the form of its R⁺ or R⁻ enantiomer, at the concentrations mentioned above for compositions containing alpha-lipoic acid by itself.

1. Use of an antioxidant, selected from the group comprising superoxide dismutases (SODs), superoxide dismutase mimetics, superoxide dismutase derivatives, racemic alpha-lipoic acid, the R⁺ enantiomer of alpha-lipoic acid, the R⁻ enantiomer of alpha-lipoic acid, and mixtures of these compounds, for the manufacture of a drug for the treatment and/or prevention of superficial eye disorders in humans or animals.

2. Use according to claim 1, characterized in that the antioxidant is an SOD or an SOD derivative or mimetic.

3. Use according to claim 2, characterized in that the SOD is a wheat SOD.

4. Use according to any one of the preceding claims, characterized in that the SOD and/or SOD derivative and/or mimetic is present in the drug at a concentration of between 1 μg/ml and 500 μg/ml, preferably of between 25 μg/ml and 50 μg/ml.

5. Use according to claim 1, characterized in that the antioxidant is racemic alpha-lipoic acid and/or its R⁺ or R⁻ enantiomer.

6. Use according to claim 5, characterized in that the antioxidant is racemic alpha-lipoic acid.

7. Use according to claim 5 or 6, characterized in that the racemic alpha-lipoic acid and/or its R⁺ or R⁻ enantiomer is present in the drug at a concentration of between 0.05 μg/ml and by 200 μg/ml, preferably of between 0.5 μg/ml and 5 μg/ml.

8. Use according to claim 1, characterized in that the antioxidant is a mixture of racemic alpha-lipoic acid and/or its R⁺ or R⁻ enantiomer, and SODs or SOD derivatives or mimetics.

9. Use according to claim 8, characterized in that the alpha-lipoic acid or its R⁺ or R⁻ enantiomer is present in the drug at a concentration of 0.05 μg/ml to 200 μg/ml, preferably of 0.5 μg/ml to 5 μg/ml, and the SOD and/or SOD derivative or mimetic is present in the drug at a concentration of between 1 μg/ml and 500 μg/ml, preferably of between 25 μg/ml and 50 μg/ml.

10. Use according to any one of the preceding claims, characterized in that the superficial eye disorders cause ocular dryness.

11. Use according to any one of claims 1 to 9, characterized in that said superficial eye disorders are a degradation of corneal cells.

12. Use according to any one of claims 1 to 9, characterized in that said eye disorders are due to exposure to environmental agents such as ozone, nitrogen oxide, sulfur dioxide, volatile organic compounds and particles released by diesel engines.

13. Use according to any one of claims 1 to 9, characterized in that the superficial eye disorders are caused by prolonged exposure to a computer screen, a television screen or a video monitor.

14. Use according to any one of claims 1 to 9, characterized in that the superficial eye disorders are due to the wearing of lenses.

15. Use according to any one of claims 1 to 9, characterized in that said eye disorders are due to exposure to salt water and/or chlorinated water.

16. Use according to any one of claims 1 to 9, characterized in that said eye disorders are due to exposure to ultraviolet A and/or B and/or C, X-rays, γ-rays or radioactive radiation.

17. Use according to any one of claims 1 to 9, characterized in that said eye disorders are due to exposure to bacteria, viruses and/or fungi.

18. Use according to any one of claims 1 to 9, characterized in that said superficial eye disorders are Gougerot-Sjögren disease, blepharitis or ocular rosacea.

19. Use according to any one of claims 1 to 9, characterized in that said superficial eye disorders are due to exposure to allergens and consist of an inflammatory reaction to degradation of the ocular surface.

20. Use according to any one of claims 1 to 10, characterized in that said eye disorders are due to exposure to one or more preservatives present in compositions administered topically to the eye.

21. Use according to claim 21, characterized in that said preservative is benzalkonium chloride.

22. Use according to any one of the preceding claims, characterized in that the drug is in the form of an eyewash, a gel or an ointment that also contains pharmaceutically acceptable excipients.
